SEQUENCE LISTING (4 sequences)

SEQ ID NO: 1 - DNA, 24 bp, Artificial
  Artificial oligonucleotide sequence.
tttttttttccattagacataacc

SEQ ID NO: 2 - DNA, 24 bp, Artificial sequence
  Artificial oligonucleotide sequence
tttttttttccattggacataacc

SEQ ID NO: 3 - DNA, 27 bp, Artificial sequence
  Artificial oligonucleotide sequence.
attcccggttatgtctaatgggtgcat

SEQ ID NO: 4 - DNA, 27 bp, Artificial sequence
  Artificial oligonucleotide sequence.
attcccggttatgtccaatgggtgcat

(19) United States
(12) Patent Application Publication
(10) Pub. No.: US 2004/0109884 A1
(43) Pub. Date: Jun. 10, 2004

(54) Polymers containing silane groups

(76) Inventors: Jens Burmeister, Koln (DE); Edgar Diessel, Koln (DE); Ingmar Dorn, Koln (DE); Burkhard Kohler, Leverkusen (DE)

Correspondence Address: KURT BRISCOE, NORRIS, MCLAUGHLIN & MARCUS, P.A., 220 EAST 42ND STREET, 30TH FLOOR, NEW YORK, NY, 10017, US

(21) Appl. No.: 10/303,403

(22) Filed: Nov. 25, 2002

(30) Foreign Application Priority Data
Nov. 28, 2001 (DE) 101 58 149.1

(51) Int. Cl. C08G 63/48; C08G 63/91; A61F 2/00

(52) U.S. Cl. 424/423; 525/54.1

(57) ABSTRACT

The invention relates to polymers containing silane groups and to the use thereof, as well as to devices coated therewith and to the use thereof.

The invention relates to polymers containing silane groups and to the use thereof, as well as to devices coated therewith and to the use thereof.

Bio- or chemosensors consist, for example, of a recognition element and an electrical or optical signal transducer. With the aid of bio- or chemosensors, it is possible to detect the presence of an analyte qualitatively or quantitatively. The functional principle of the sensors is based on the recognition reaction between the recognition element and the analyte to be detected. Examples of recognition reactions are the binding of ligands to complexes, the sequestration of ions, the binding of ligands to (biological) receptors, membrane receptors or ion channels, of antigens or haptens to antibodies, of substrates to enzymes, of DNA or RNA to specific proteins, of aptamers or “spiegelmers” to their targets, the hybridization of DNA/RNA/PNA or other nucleic acid analogues, or the processing of substrates by enzymes. The recognition element is, for example, immobilized covalently or non-covalently on the surface of a signal transducer. Examples of analytes are DNA, RNA, PNA, nucleic acid analogues, enzyme substrates, peptides., proteins, potential active agents, medicaments, cells, viruses. Examples of recognition elements are DNA, RNA, PNA, nucleic acid analogues, aptamers, “spiegelmers”, peptides, proteins, sequestrants for metals/metal ions, cyclodextrins, crown ethers, antibodies or fragments thereof, anticalines, enzymes, receptors, membrane receptors, ion channels, cell adhesion proteins, gangliosides, mono- or oligosaccharides.

Bio- or chemosensors can be used in environmental analysis, the food industry, human and veterinary diagnosis, crop protection, and in biochemical research, in order to determine analytes qualitatively and/or quantitatively. If a variety of detector elements are bound, while being spatially separated from one another, to the surface of the signal transducer, then a large number of recognition reactions with a sample to be studied can be analysed simultaneously. This is implemented, for example, in so-called DNA arrays, in which various DNA sequences (for example oligonucleotides or cDNAs) are immobilized on a solid substrate (for example glass). Such DNA arrays can be read by using optical or electrical methods, and they are employed in expression profiling, sequencing, detection of viral or bacterial nucleic acids, genotyping, etc.

The recognition reaction of bio- or chemosensors may be detected, for example, by using optical, electrical, mechanical and/or magnetic detection methods, in which biological recognition molecules are immobilized on dielectric surfaces.

Optical detection methods are based, for example, on the detection of fluorescently labelled biomolecules on dielectric surfaces. The fluorescence may in this case be excited by means of planar optical waveguides, Duveneck et al. U.S. Pat. No. 5,959,292 (1999), total reflection at interfaces, Katerkamp DE 196 28 002, or on the surface of optical fibres, Hirschfeld U.S. Pat. No. 4,447,546. The binding of a target molecule to a detector molecule, which is immobilized on a waveguide, may nevertheless be detected without labelling by means of the change in the optical refractive index: grating coupler: Tiefenthaler et al., U.S. Pat. No. 4,815,843, Kunz, U.S. Pat. No. 5,442,169, interferometer: Stamm et al., Sens. & Act. B 11, 177 (1993), Schipper et al., Anal. Chem. 70(6), 1192 (1998), resonant mirror: Cush et al., Biosensors & Bioelectronics 8, 347 (1993), multilayered grating resonance: Yang et al., Real-time monitoring of small molecule-protein interaction by a Multilayered Grating Resonance (MGR) Biosensor, Biosensors 2000, San Diego (2000). Detection in which interferences on dielectric films are utilized is also carried out without labelling: reflectometric interference spectroscopy: Gauglitz et al., Sens. & Act. B 11, 21 (1993) or ellipsometry: Striebel et al. Biosens. & Bioelectr. 9, 139 (1994). An alternative method is enzymatically induced film formation, which is evaluated interferometrically: Jenison, Clin. Chem. 47, 1894 (2001).

A new class of electrical biosensors is based on the detection of analytes which are labelled by metallic particles, for example nanoparticles. For detection, these particles are enlarged, by autometallographic deposition, until they short-circuit a microstructured circuit. This is demonstrated by a simple direct-current impedance measurement. The fundamental patents for this are held by Molecular Circuitry Inc. (MCI), King of Prussia, Pa., USA (U.S. Pat. No. 4,794,089; U.S. Pat. No. 5,137,827; U.S. Pat. No. 5,284,748). The detection of nucleic acids by direct-current impedance measurement has recently been demonstrated (Möller et al., Langmuir 2001). The detector DNA was in this case immobilized by using an alklylsilane. To date, there is no report of the differentiation of DNA sequences, which differ by only one base in their sequence, by direct-current impedance measurement. The differentiation of DNA sequences, which differ by only one base in their sequence, by a gold-labelled DNA detector sample using optical means has, however, recently been described (Taton et al., Science 2000, 289, 1757-1760).

Field-effect transistors can be used as electronic transducers, for example for an enzymatic reaction: Zayats et al., Biosens. & Bioelectron. 15, 671 (2000).

As mechanical transducers, oscillating quartzes are described, in which the resonant frequency is changed by mass buildup: Steinem et al., Biosens. & Bioelectronics 12, 787 (1997). In an alternative mechanical transducer, surface acoustic waves that are modified by target adsorption are excited in interdigital structures, Howe et al., Biosens. & Bioelectron. 15, 641 (2000).

If the target molecules are labelled with magnetic beads, then the recognition reaction can be detected by means of the magnetic effect of the bead on the giant magnetic resistance (GMR) of a corresponding resistor: Baselt et al., Biosens. & Bioelectron. 13, 731 (1998).

Detector elements can be coupled covalently or non-covalently to the surface of the signal transducer. Covalent immobilization of recognition elements, for example of DNA, on sensor surfaces has decisive advantages, in terms of stability, reproducibility and specificity of the coupling, over non-covalent coupling. A review of methods for preparing DNA-coated surfaces is given by S. L. Beaucage, Curr. Med. 2001, 8, 1213-1244.

An example of non-covalent coupling is the spotting of cDNA on glass substrates, on which polylysine has been adsorbed beforehand. This method is very widespread in the production of DNA microarrays. By functionalizing surfaces with silanes, for example aminoalkylsilanes, a monolayer of amino groups can be covalently applied to the sensor surface. The amino groups can be activated by difunctional linkers to which, for example, amino-modified DNA can then be covalently coupled. Alternatively, the DNA may be suitably activated and subsequently bound to the surface, which has been functionalized with aminoalkyl groups. This method is described, for example, B. Joos, H. Kuster, R. Core, Anal. Biochem. 1997, 247, 96-101. A disadvantage of such a method, however, is the fact that the maximum achievable DNA density is limited by the available monolayer. There is a need for such methods of functionalizing surfaces which make it possible to immobilize a significantly higher number of detector elements per unit area than is possible with a monolayer. A higher density of detector elements improves the signal/noise ratio as well as the dynamic range of the sensor. One possible solution to the said problem is the formation of dendrimer-like structures in a synthesis comprising a plurality of steps. This method is described, for example, in M. Beier, J. Hoheisel, Nucl. Acids Res. 1999, 27, 1970-1977. Another proposed solution method, for example, is the coating of gold surfaces with thiol-carboxylic acids, which are subsequently activated and covalently linked in aqueous solution with poly-L-lysine (Frey, B. L., Corn, R. M. Anal. Chem. 1996, 68, 3187). Glass surfaces can be coated with a layer of a polyacrylamide gel. The free amide groups of the polymer can be reacted with hydrazine, which permits immobilization of the amino-modified biomolecules onto the resulting acid hydrazide groups. This method is described, for example, in: Khrapko K. R. et al., FEBS Lett. 1989, 256, 118 and in Khrapko K. R. et al., DNA Sequence 1991, 1, 375. Before production of the polyacrylamide gel on the biochip surface, acrylamide groups can be bound to the surface via suitable functional silanes. Copolymerization of N,N-dimethyl acrylamide and N-(5,6-di-O-isopropylidene)hexyl acrylamide in the presence of N,N-methylene-bis-acrylamide and ammonium persulphate on acrylamido-silanized glass substrates leads, after removal of the protective groups, to an aldehyde-functionalized gel which can be reacted with amino-functionalized detector elements (Timofeev, E. N., Kochetskova, S. V., Mirzabekov, A. D., Florentiev, V. L., Nucl. Acids Res. 1996, 24, 3142). A simple process which would make it possible to covalently coat a sensor surface, in one reaction step, with a polymer suitable for the biofunctionalization has not yet been described.

Patent Application EP 0596421 A1 in the name of the company Hoffmann-La Roche describes silanes of the general form (R1R2R3)Si—X—Y and their use for producing optical biosensors. Claim 3 describes Y as a polymer from the group of oligovinyl alcohols, oligoacrylic acids, oligoacrylic derivatives, oligoethylene glycols or polysaccharides. Reference is not made to silylated polyamines, for example polylysin, and their use for producing electrical biosensors. Application EP 0596421 was withdrawn. The company Hoffmann-La Roche later filed the European patent EP 0653 429 A1, in which reference to polymers is no longer made.

Hyperbranched copolyamides have been produced by reacting, for example, L-lysine and ε-caprolactam (WO 00/68298). Such branched copolyamides have been used to improve the properties of thermoplastic materials. Subsequent silylation of these polymers has not been carried out.

The silylation of L-lysine is described in Beauregard, G. P. et al., J. Appl. Polym. Sci. 2001, 79, 2264-2271. The silylation was carried out with bis(trimethylsilyl)acetamide, and it led to an improvement of the solubility of the polymer in organic solvents. Trimethylsilyl groups are not suitable for enabling covalent coating with an oxidic surface. In the context of developing pH-sensitive drug delivery systems, WO 00/75164 describes the silylation of polylysine with 3-aminopropyltriethoxysilane. During this silylation, direct linkage of the silane to the ε-amino groups of polylysine takes place, with a silazane being formed, so that a polymer produced in this way cannot be used for the covalent coating of surfaces.

It is an object of the invention to modify (coat) surfaces of biosensors in such a way as to permit binding of detector elements, for example nucleic acids. A method is to be provided which permits covalent, specific binding of, for example, nucleic acids on planar surfaces, for example consisting of glass or silicon dioxide. The detector elements should, in particular, be bonded in such a way as to permit electrical detection of nucleic acid targets on unstructured or laterally structured surfaces. In particular, the electrical detection of nucleic acid targets, on the basis of the specific coupling of the detector nucleic acid, should take place so selectively as to permit differentiation of nucleic acid target sequences which differ by only one base in their sequence. The material to be provided for the coating of sensor surfaces must furthermore meet the following stringent requirements:

The coating process must be as simple as possible, that is to say it must comprise the fewest possible steps. In the ideal case, the coating process should comprise only one step.

The immobilization of the recognition elements must be stable under the reaction conditions of the recognition reaction.

The functionality of the recognition elements must still be present after the immobilization.

So that only the specific recognition reaction is detected by the signal transducer, any kind of non-specific binding to the signal transducer surface must be suppressed.

In order to achieve a high signal/noise ratio and a high selectivity of the recognition reaction, according to the prior art it is necessary to achieve a surface density of bound recognition elements which is greater than one monolayer.

The invention relates to a hyperbranched silane-functional polyamide-urethane, which can be obtained by condensation of

A) from 40 to 100 parts by weight, preferably from 60 to 90 parts by weight, of one or more amino acids having at least two amino groups and one carboxyl group and/or lactams thereof, for example L-lysine, D-lysine, a-L-amino-ε-caprolactam, α-D-amino-ε-caprolactam, 3,5-diaminobenzoic acid, 2,4-diaminobenzoic acid or mixtures of these monomers, preferably L-lysine,

B) from 0 to 60 parts by weight, preferably from 5 to 20 parts by weight, of one or more amino acids having one amino group and one carboxyl group and/or lactams thereof, for example ε-caprolactam, laurinlactam, 6-aminocaproic acid, 11-aminoundecanoic acid or mixtures thereof, preferably ε-caprolactam, and

C) from 0 to 60 parts by weight, preferably from 5 to 20 parts by weight, of diamines of Formula (I),

H₂N—R—NH₂ (I)

in which

R is a C₂-C₃₆ alkylene or cycloalkylene radical, a C₈-C₂₀ alkylenearylene radical, or a radical of Formula (II),

—R1(-X—CH₂—C(R2)H—)ₙ—X—R1- (II)

in which

R1 is an ethylene, propylene or butylene radical,

R2 is a methyl group or a hydrogen atom, preferably a hydrogen atom,

X is an oxygen atom or an NH group, and

n is a natural number from 1 to 100,

particularly preferably 1,6-diaminohexane, IPDA or bis(4-aminocyclohexyl)methane,

in the melt, preferably at temperatures of 160-260° C., in the presence or absence of phosphorus-containing catalysts, advantageously in the presence of from 0.1 to 1 part by weight of triphenyl phosphite, and

subsequent reaction of the melt condensation product of the structural units A and optionally B and/or C, preferably at temperatures of 0-100° C., with from 1 to 20% by weight, advantageously from 5 to 15% by weight, expressed in terms of the melt condensation product, of an isocyanatosilane of Formula (III),

O═C═N—CH₂—CH₂—CH₂—Si(OR4)₃ (III)

in which

R4 is a C₁-C₄ alkyl radical or a methoxyethyl radical,

wherein the melt condensation product and/or the isocyanatosilane may be pre-dissolved in a dipolar-aprotic solvent, for example DMF, DMA, NMP or DMSO.

The hyperbranched silane-functional polyamide-urethane according to the invention is suitable for the coating of surfaces, in particular oxidic surfaces such as are used, for example, as sensor surfaces for electrical or optical signal transducers. The coating of the sensor surface with the polymer is carried out in one reaction step.

The invention also relates to a device having at least one surface coated with a polyamide-urethane according to the invention, for example a signal transducer, in particular an electrical, optical, magnetic and/or mechanical signal transducer, with a coating of this polymer. Biological, chemical or biochemical recognition elements, for example DNA, RNA, aptamers, receptors etc., are bound to the surfaces coated with the polymer. The (bio)functionalized surfaces are employed in sensor technology, and they are an essential constituent part of bio- or chemosensors, for example as biochips which can be read by using electrical or optical methods. The oxidic surfaces coated with the polymer are, in particular, suitable for immobilizing detector nucleic acids covalently on the surface. The so-called detector nucleic acids immobilized in this way are, in particular, suitable for differentiating by electrical detection between nucleic acids which differ by only one base in their sequence.

One of the two amino groups, or both amino groups, of component A may be made to react with amine formation during the melt condensation, the result being a hyperbranched polyamide, some of whose excess amino groups are reacted with the isocyanatosilane to form urea groups. Formula (IV) shows, by way of example, one of the possible units of a silane-functional polyamide-urethane according to the invention (* represents continuation of the polymer):

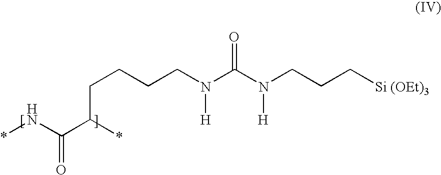

The amino groups of the polymer are suitable for the binding of recognition elements directly or with the aid of a crosslinker covalently, coordinatively or via another chemical bond onto the polymer. The direct coupling of the recognition elements can be carried out before or after the sensor surface is coated with the polymer. All homo- or heterodifunctional amine-group-reactive compounds known according to the prior art, for example bis-isothiocyanates, bis-isocyanates, bis-N-hydroxysuccinimide esters, bis-sulpho-N-hydroxysuccinimide esters, bis-imidic acid esters, etc. may be used as crosslinkers.

The hyperbranched silane-functional polyamide-urethane according to the invention has the following advantages over compounds known according to the prior art for the coating of sensor surfaces:

The coating of sensor surfaces with the silane-functional polyamide-urethane is carried out in a single reaction step.

A particularly high density of detector elements is achieved by the coating of sensor surfaces with the silane-functional polyamide-urethane and subsequent coupling of detector elements, for example nucleic acids.

The high density of detector elements achieved by the coating of sensor surfaces with the silane-functional polyamide-urethane and subsequent covalent coupling of nucleic acids makes it possible, by direct-current impedance measurement, to differentiate nucleic acid targets which differ by only one base with respect to their sequence.

In contrast to pure poly-lysine, which contains only alpha-amino acids, the hyperbranched polyamide is soluble in organic solvents, so that derivative formation, for example with isocyanatosilanes, is made possible for the first time.

The silane-functional polyamide-urethane can be applied from organic solvents, which facilitates handling. This dissolving behaviour is also advantageous since certain silane functions, for example the trialkoxysilane functional group, are stable only in organic solvents. In contrast thereto, poly-L-lysine is water-soluble only in salt form, which makes it impossible to form derivatives with isocyanatosilanes. It can therefore be anchored to the surface only electrostatically.

The silane-functional, hyperbranched polyamide-urethanes, in contrast to dendrimers, can be produced in a one-pot reaction in two steps, polycondensation and subsequent reaction with isocyanatosilane. The structural units are readily available technically. Through expedient structural-unit selection, in contrast to biopolymers, the properties can be varied in a straightforward way.

Compared with polysaccharides, polyamides have the advantage that many primary amino groups are available as reactive linkage points for the subsequent chemistry. Chitosan, the only readily available amino-functional polysaccharide, is barely soluble in organic solvents, so that similar disadvantages arise as in the case of poly-L-lysine. With the silane-functional, hyperbranched polyamide-urethanes, the density of the ami groups can be adjusted in a controlled way through structural-unit selection. Polysaccharides are overfunctionalized with respect to OH groups, these OH groups being capable of esterifying slowly to form trialkoxy groups after silanization, so that undesired crosslinking may occur. For the subsequent chemistry, the OH groups are less well suited than amino groups. In the case of silanes, Si—O bonds are more stable than Si—N bonds, so that silanized polyamide-urethanes with an excess of amino groups are comparatively storage-stable. Furthermore, the amide groups assist adhesion to oxidic surfaces by particularly stable hydrogen bridge bonds, which is an advantage over polysachharides.

Polymers per se have the advantage, over monomolecular silanization reagents, of multifunctionality, so that adhesion to undersurfaces as well as linkage of further biomolecules is directly favoured on entropic grounds.

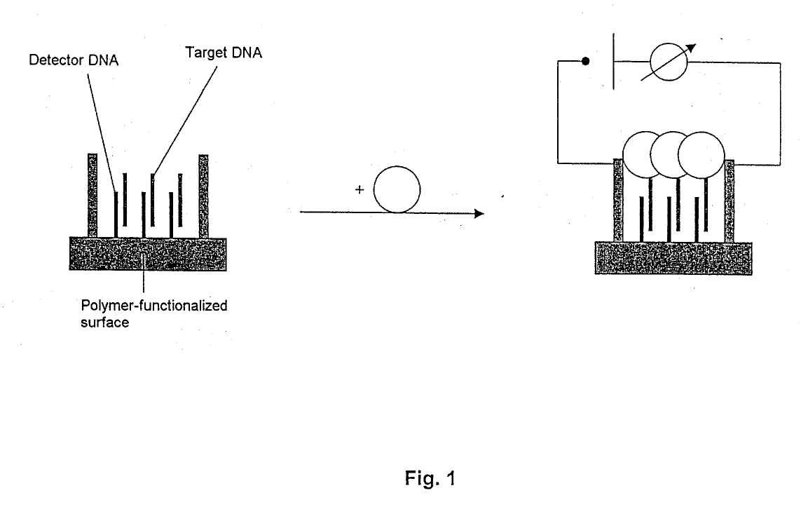

The invention will be explained in more detail below with reference to a drawing (FIG. 1) and exemplary embodiments.

FIG. 1: schematic structure of a biosensor with direct-current impedance measurement.

### EXAMPLES

### Example 1

### Production of a Silane-Functional Polyamide-Urethane

200 g of L-lysine, 50 g of ε-caprolactam, 50 g of 1,6-diaminohexane and 0.5 g of TPP were made to react at 240° C.; water was distilled off. The resulting polyamide was diluted in the ratio 8:1 with NMP. 9 g of the polymer were reacted for silanization for 2 h under an N₂ atmosphere with 0.1 g of triethoxysilylpropyl isocyanate at RT (room temperature=approximately 20° C.); the silane reacted via urethane groups with the amino groups of the polyamide.

### Example 2

### Coating of Surfaces with a Silane-Functional Polyamide-Urethane

Structured or unstructured chips of glass or oxidized silicon were treated for 30 min with argon-induced plasma at standard pressure, and subsequently heated for 5 min to 80° C. A 1% strength solution of the silane-functional polyamide-urethane in a mixture of acetone/DMF/water (volume ratio 7.5:2:0.5 v/v/v) was incubated for 15 min at room temperature with the purified chip. After functionalization, the surfaces were washed with acetone and subsequently dried for 45 min at 110° C.

### Example 3

### Coupling of Detector Nucleic Acids to Functionalized Surfaces

Detector DNA A (5′-amino-TTT TTT TTT CCA TTA GAC ATA ACC) and detector DNA G (5′-amino-TTT TTT TTT CCA TTG GAC ATA ACC) were dissolved in phosphate buffer pH 7.2 and respectively incubated with 0.1M of bis-sulpho-succinimidyl suberate (BS3) for 10 min at RT. The reaction was terminated by dilution with phosphate buffer. The detector DNAs were purified by chromatography on a NAP-10 column (Pharmacia). The purified detector DNAs were applied in volumes of, for example, 25 μl, onto the silanized surfaces, and incubated overnight at RT. The resulting DNA chips were washed with a 1% strength ammonium hydroxide and water, and subsequently dried at RT. The unreacted amino groups on the chip surface were blocked by incubation with 0.4 mg/ml of BS3 in 0.1 M phosphate buffer pH 7.2.

### Example 4

### Conduct of DNA Hybridization Reactions and Gold Labelling

Hybridisation reactions were then carried out on the structured or unstructured surfaces coated with polymer and detector DNA; all four possible combinations were studied: detector DNA A+target DNA T (5′-biotin-ATT CCC GGT TAT GTC TAA TGG GTG CAT), detector DNA A+target DNA C (5′-biotin-ATT CCC GGT TAT GTC CAA TGG GTG CAT), detector DNA G+target DNA C and detector DNA G+target DNA T (abbreviated to AT/AC/GC/GT). To that end, 10-7M solutions of the respective target DNA in Tris buffer pH 7.2 were incubated with the chip for 3 h at 42° C. Washing was then carried out with Tris buffer. The hybridized target DNAs were incubated for 1 h at RT with a solution of streptavidin-gold (diameter of the gold particles 25 nm, company Aurion, Netherlands). The chips were washed with water and subsequently dried at RT. The gold-labelled nucleic acids were treated 3× for 15 min with the enhancer solution from the company Biocell (Biocell L 15) and subsequently dried.

### Example 5

### Direct-Current Impedance Measurement on Gold-Labelled Nucleic Acid Targets

The direct-current impedance measurement of the enhanced chip surfaces may be carried either between externally applied gold electrodes or between evaporation-coated gold electrodes (structured surfaces). The direct-current impedance measurement between externally applied electrodes showed that, in the case of the “matching” combinations GC and AT, impedances <5 kΩ were measured over a distance of 80 μm, whereas the combinations GT and AC showed impedances >100 MΩ even over a distance of 10 μm. During the direct-current impedance measurement between evaporation-coated electrodes, it was found that impedances <5 kΩ were measured with an electrode spacing of 20 μm in the case of the combinations GC and AT, whereas impedances >100 MΩ were measured for the combinations AC and GT down to an electrode spacing of 10 μm.

1. Hyperbranched silane-functional polyamide-urethane, which can be obtained by condensation of
A) from 40 to 100 parts by weight of one or more amino acids having at least two amino groups and one carboxyl group and/or lactams thereof,
B) from 0 to 60 parts by weight of one or more amino acids having one amino group and one carboxyl group and/or lactams thereof, and
C) from 0 to 60 parts by weight of diamines of Formula (I),
H₂N-R-NH₂ (I)
in which
R stands for a C2-C36 alkylene or cycloalkylene radical, a C8-C20 alkylenearylene radical, or a radical of Formula (II),
—R1(-X—CH₂—C(R2)H—)ₙ—X—R1- (II)
in which
R1 is an ethylene, propylene or butylene radical,
R2 is a methyl group or a hydrogen atom,
X is an oxygen atom or an NH group, and
n is a natural number from 1 to 100,
in the melt and subsequent reaction of the melt condensation product with from 1 to 20% by weight, expressed in terms of the melt condensation product, of an isocyanatosilane of Formula (III),
O═C═N—CH₂—CH₂—CH₂—Si(OR4)₃ (III)
in which
R4 is a C₁-C₄ alkyl radical or a methoxyethyl radical.

2. A method of coating a surface comprising coating said surface with the polyamide-urethane according to claim 1.

3. Method according to claim 2, wherein the polyamide-urethane is coated onto an oxidic surface.

4. Device having at least one surface coated with a polyamide-urethane according to claim 1.

5. Device according to claim 4, wherein the device is a signal transducer.

6. Device according to claim 4, wherein one or more detector nucleic acids and/or one or more antibodies are covalently bonded to the polyamide-urethane.

7. Device according to claim 6, wherein one or more detector nucleic acids are bonded to the polyamide-urethane.

8. Device according to claim 4, wherein the device is an array.

9. Method for the differentiation of nucleic acids which differ by only one base in their sequence, said method comprising differentiating said nucleic acids on the device according to claim 7.

10. Method according to claim 9, wherein the differentiation of nucleic acids is carried out by direct-current impedance measurement.

11. Method according to claim 5, wherein the signal transducer is an optical, electrical, mechanical and/or magnetic signal transducer.

12. Device according to claim 8, wherein the array is a DNA array or a protein array.
